(19) United States
(12) Patent Application Publication
(10) Pub. No.: US 2009/0198274 A1
(43) Pub. Date: Aug. 6, 2009

(54) METHOD AND APPARATUS FOR RE-ATTACHING THE LABRUM OF A HIP JOINT

(76) Inventors: Matthew Frushell, Danville, CA (US); Bryan Kelly, New York, NY (US); Jonathan Dewey, Sunnyvale, CA (US); James R. Flom, San Carlos, CA (US); Chris Pamichev, Sunnyvale, CA (US)

Correspondence Address: Mark J. Pandiscio;Pandiscio & Pandiscio, P.C., 470 Totten Pond Road, Waltham, MA, 02451-1914, US

(21) Appl. No.: 12/235,416

(22) Filed: Sep. 22, 2008

Related U.S. Application Data
(60) Provisional application No. 60/994,576, filed on Sep. 20, 2007.

(51) Int. Cl. A61B 17/04

(52) U.S. Cl. 606/232

(57) ABSTRACT

A system for securing soft tissue to bone, the system comprising:
a center post anchor comprising a body adapted for disposition in bone and having a retention element thereon for retaining the body in bone, the center post anchor comprising a suture having a first portion secured to the body and a second portion residing free of the body and adapted to be passed through the soft tissue which is to be secured to the bone; and
a bridge post anchor comprising a body adapted for disposition in bone and having a retention element thereon for retaining the body in bone, the bridge post anchor including a capture element for capturing the second portion of the suture to the bone, such that when the center post anchor is disposed in bone and the second portion of the suture extends through in bone can secure the soft tissue to the bone.

### REFERENCE TO PENDING PRIOR PATENT APPLICATION

This patent application claims benefit of pending prior U.S. Provisional Patent Application Ser. No. 60/994,576, filed Sep. 20, 2007 by Brian Kelly et al. for METHOD AND APPARATUS FOR RE-ATTACHING THE LABRUM OF A HIP JOINT (Attorney's Docket No. FIAN-9 PROV), which patent application is hereby incorporated herein by reference.

### FIELD OF THE INVENTION

This invention relates to surgical methods and apparatus in general, and more particularly to methods and apparatus for treating the hip joint.

### BACKGROUND OF THE INVENTION

### The Hip Joint in General

The hip joint is a ball-and-socket joint which movably connects the leg to the torso. The hip joint is capable of a wide range of different motions, e.g., flexion and extension, abduction and adduction, medial and lateral rotation, etc. See FIGS. 1A, 1B, 1C and 1D.

With the possible exception of the shoulder joint, the hip joint is perhaps the most mobile joint in the body. Significantly, and unlike the shoulder joint, the hip joint carries substantial weight loads during most of the day, in both static (e.g., standing and sitting) and dynamic (e.g., walking and running) conditions.

The hip joint is susceptible to a number of different pathologies. These pathologies can have both congenital and injury-related origins. In some cases, the pathology can be substantial at the outset. In other cases, the pathology may be minor at the outset but, if left untreated, may worsen over time. More particularly, in many cases, an existing pathology may be exacerbated by the dynamic nature of the hip joint and the substantial weight loads imposed on the hip joint.

The pathology may, either initially or thereafter, significantly interfere with patient comfort and lifestyle. In some cases, the pathology can be so severe as to require partial or total hip replacement. A number of procedures have been developed for treating hip pathologies short of partial or total hip replacement, but these procedures are generally limited in scope due to the significant difficulties associated with treating the hip joint.

A better understanding of various hip joint pathologies, and also the current limitations associated with their treatment, can be gained from a more thorough understanding of the anatomy of the hip joint.

### Anatomy of the Hip Joint

The hip joint is formed at the junction of the femur and the hip. More particularly, and looking now at FIG. 2, the ball of the femur is received in the acetabular cup of the hip, with a plurality of ligaments and other soft tissue serving to hold the bones in articulating condition.

More particularly, and looking now at FIG. 3, the femur is generally characterized by an elongated body terminating, at its top end, in an angled neck which supports a hemispherical head (also sometimes referred to as “the ball”). As seen in FIGS. 3 and 4, a large projection known as the greater trochanter protrudes laterally and posteriorly from the elongated body adjacent to the neck of the femur. A second, somewhat smaller projection known as the lesser trochanter protrudes medially and posteriorly from the elongated body adjacent to the neck. An intertrochanteric crest (FIGS. 3 and 4) extends along the periphery of the femur, between the greater trochanter and the lesser trochanter.

Looking next at FIG. 5, the hip socket is made up of three constituent bones: the ilium, the ischium and the pubis. These three bones cooperate with one another (they typically ossify into a single “hip bone” structure around the age of 25 or so) so as to collectively form the acetabular cup. The acetabular cup receives the head of the femur.

Both the head of the femur and the acetabular cup are covered with a layer of articular cartilage which protects the underlying bone and facilitates motion. See FIG. 6.

Various ligaments and soft tissue serve to hold the ball of the femur in place within the acetabular cup. More particularly, and looking now at FIGS. 7 and 8, the ligamentum teres extends between the ball of the femur and the base of the acetabular cup. As seen in FIG. 9, a labrum is disposed about the perimeter of the acetabular cup. The labrum serves to increase the depth of the acetabular cup and effectively establishes a suction seal between the ball of the femur and the rim of the acetabular cup, thereby helping to hold the head of the femur in the acetabular cup. In addition to the foregoing, and looking now at FIG. 10, a fibrous capsule extends between the neck of the femur and the rim of the acetabular cup, effectively sealing off the ball-and-socket members of the hip joint from the remainder of the body. The foregoing structures (i.e., the ligamentum teres, the labrum and the fibrous capsule) are encompassed and reinforced by a set of three main ligaments (i.e., the iliofemoral ligament, the ischiofemoral ligament and the pubofemoral ligament) which extend between the femur and the perimeter of the hip socket. See FIGS. 11 and 12.

### Pathologies of the Hip Joint

As noted above, the hip joint is susceptible to a number of different pathologies. These pathologies can have both congenital and injury-related origins.

By way of example but not limitation, one important type of congenital pathology of the hip joint involves impingement between the neck of the femur and the rim of the acetabular cup. In some cases, and looking now at FIG. 13, this impingement can occur due to irregularities in the geometry of the femur. This type of impingement is sometimes referred to as a cam-type femoroacetabular impingement (i.e., a cam-type FAI). In other cases, and looking now at FIG. 14, the impingement can occur due to irregularities in the geometry of the acetabular cup. This latter type of impingement is sometimes referred to as a pincer-type femoroacetabular impingement (i.e., a pincer-type FAI). Impingement can result in a reduced range of motion, substantial pain and, in some cases, significant deterioration of the hip joint.

By way of further example but not limitation, another important type of congenital pathology of the hip joint involves defects in the articular surface of the ball and/or the articular surface of the acetabular cup. Defects of this type sometimes start fairly small but often increase in size over time, generally due to the dynamic nature of the hip joint and also due to the weight-bearing nature of the hip joint. Articular defects can result in substantial pain, induce and/or exacerbate arthritic conditions and, in some cases, cause significant deterioration of the hip joint.

By way of further example but not limitation, one important type of injury-related pathology of the hip joint involves trauma to the labrum. More particularly, in many cases, an accident or sports-related injury can result in the labrum being torn away from the rim of the acetabular cup, typically with a tear running through the body of the labrum. See FIG. 15. These types of injuries can be very painful for the patient and, if left untreated, can lead to substantial deterioration of the hip joint.

### The General Trend Toward Treating Joint Pathologies Using Minimally-Invasive, and Earlier, Interventions

The current trend in orthopedic surgery is to treat joint pathologies using minimally-invasive techniques. Such minimally-invasive, “keyhole” surgeries generally offer numerous advantages over traditional, “open” surgeries, including reduced trauma to tissue, less pain for the patient, faster recuperation times, etc.

By way of example but not limitation, it is common to re-attach ligaments in the shoulder joint using minimally-invasive, “keyhole” techniques which do not require laying open the capsule of the shoulder joint. By way of further example but not limitation, it is common to repair torn meniscal cartilage in the knee joint, and/or to replace ruptured ACL ligaments in the knee joint, using minimally-invasive techniques.

While such minimally-invasive approaches can require additional training on the part of the surgeon, such procedures generally offer substantial advantages for the patient and have now become the standard of care for many shoulder joint and knee joint pathologies.

In addition to the foregoing, in view of the inherent advantages and widespread availability of minimally-invasive approaches for treating pathologies of the shoulder joint and knee joint, the current trend is to provide such treatment much earlier in the lifecycle of the pathology, so as to address patient pain as soon as possible and so as to minimize any exacerbation of the pathology itself. This is in marked contrast to traditional surgical practices, which have generally dictated postponing surgical procedures as long as possible so as to spare the patient from the substantial trauma generally associated with invasive surgery.

### Treatment for Pathologies of the Hip Joint

Unfortunately, minimally-invasive treatments for pathologies of the hip joint have lagged far behind minimally-invasive treatments for pathologies of the shoulder joint and knee joint. This is generally due to (i) the constrained geometry of the hip joint itself, and (ii) the nature and location of the pathologies which must typically be addressed in the hip joint.

More particularly, the hip joint is generally considered to be a “tight” joint, in the sense that there is relatively little room to maneuver within the confines of the joint itself. This is in marked contrast to the shoulder joint and the knee joint, which are generally considered to be relatively “spacious” joints (at least when compared to the hip joint). As a result, it is relatively difficult for surgeons to perform minimally-invasive procedures on the hip joint.

Furthermore, the pathways for entering the interior of the hip joint (i.e., the pathways which exist between adjacent bones) are generally much more constraining for the hip joint than for the shoulder joint or the knee joint. This limited access further complicates effectively performing minimally-invasive procedures on the hip joint.

In addition to the foregoing, the nature and location of the pathologies of the hip joint also complicate performing minimally-invasive procedures on the hip joint. By way of example but not limitation, consider a typical detachment of the labrum in the hip joint. In this situation, instruments must generally be introduced into the joint space using an angle of approach which is set at approximately a right angle to the angle of re-attachment. This makes drilling into bone, for example, much more complicated than where the angle of approach is effectively aligned with the angle of re-attachment, such as is frequently the case in the shoulder joint. Furthermore, the working space within the hip joint is typically extremely limited, further complicating repairs where the angle of approach is not aligned with the angle of re-attachment.

As a result of the foregoing, minimally-invasive hip joint procedures are still relatively difficult to perform and relatively uncommon in practice. Consequently, patients are typically forced to manage their hip pain for as long as possible, until a resurfacing procedure or a partial or total hip replacement procedure can no longer be avoided. These procedures are generally then performed as a highly-invasive, open procedure, with all of the disadvantages associated with highly-invasive, open procedures.

As a result, there is, in general, a pressing need for improved methods and apparatus for treating pathologies of the hip joint.

### Re-Attaching the Labrum of the Hip Joint

As noted above, hip arthroscopy is becoming increasingly more common in the diagnosis and treatment of various hip pathologies. However, due to the anatomy of the hip joint and the pathologies associated with the same, hip arthroscopy is currently practical for only selected pathologies and, even then, hip arthroscopy has generally met with limited success.

One procedure which is sometimes attempted arthroscopically relates to the repair of a torn and/or detached labrum. This procedure may be attempted (i) when the labrum has been damaged but is still sufficiently healthy and intact as to be capable of repair and/or re-attachment, and (ii) when the labrum has been deliberately detached (e.g., so as to allow for acetabular rim trimming to treat a pathology such as a pincer-type FAI) and needs to be subsequently re-attached. See, for example, FIG. 16, which shows a normal labrum L secured to the acetabulum A, and FIG. 17, which shows labrum L detached from acetabulum A. In this respect it should also be appreciated that repairing the labrum rather than removing the labrum is generally desirable, inasmuch as studies have shown that patients whose labrum has been repaired tend to have better long-term outcomes than patients whose labrum has been removed.

Unfortunately, current methods and apparatus for arthroscopically re-attaching the labrum are somewhat problematic. The present invention is intended to improve upon the current approaches for labrum re-attachment.

More particularly, current approaches for arthroscopically re-attaching the labrum typically use apparatus originally designed for use in re-attaching ligaments to bone. For example, one such approach utilizes a screw-type bone anchor, with two sutures extending therefrom, and involves deploying the bone anchor in the acetabulum above the labrum re-attachment site. A first one of the sutures is passed either through the detached labrum or, alternatively, around the detached labrum. Then the first suture is tied to the second suture so as to support the labrum against the acetabular rim.

Since the suture knot typically stands proud of the adjacent tissue, the surgeon generally tries to position the knot above the acetabular rim, exterior to the articulating surface of the hip joint, so as to avoid abrasion during hip motion. However, this can be difficult to achieve, given the limited space within the hip joint, the angle of approach dictated by the patient's anatomy, etc. Indeed, the mere act of arthroscopically tying a suture knot can be relatively complex and time-consuming. Thus, the need to precisely position the knot outside the articulating portion of the joint can further complicate an already-difficult arthroscopic procedure.

Accordingly, a primary object of the present invention is to simplify the foregoing procedure by providing a new approach for arthroscopically re-attaching the labrum to the acetabulum.

### SUMMARY OF THE INVENTION

The present invention provides a new approach for arthroscopically re-attaching the labrum to the acetabulum.

Significantly, this new approach does not require the tying of knots in order to re-attach the labrum to the acetabulum.

More particularly, the present invention provides a novel method and apparatus for knotlessly re-attaching the labrum to the acetabulum. As a result, the present invention provides a simpler, faster and more convenient approach for securing the labrum to the acetabulum.

In one preferred form of the present invention, there is provided a system for securing soft tissue to bone, the system comprising:

a center post anchor comprising a body adapted for disposition in bone and having a retention element thereon for retaining the body in bone, the center post anchor comprising a suture having a first portion secured to the body and a second portion residing free of the body and adapted to be passed through the soft tissue which is to be secured to the bone; and

a bridge post anchor comprising a body adapted for disposition in bone and having a retention element thereon for retaining the body in bone, the bridge post anchor including a capture element for capturing the second portion of the suture to the bone, such that when the center post anchor is disposed in bone and the second portion of the suture extends through the soft tissue, disposition of the bridge post anchor in bone can secure the soft tissue to the bone.

In another form of the present invention, there is provided a method for securing soft tissue to bone, the method comprising:

providing a system comprising:
a center post anchor comprising a body adapted for disposition in bone and having a retention element thereon for retaining the body in bone, the center post anchor comprising a suture having a first portion secured to the body and a second portion residing free of the body and adapted to be passed through the soft tissue which is to be secured to the bone; and
a bridge post anchor comprising a body adapted for disposition in bone and having a retention element thereon for retaining the body in bone, the bridge post anchor including a capture element for capturing the second portion of the suture to the bone, such that when the center post anchor is disposed in bone and the second portion of the suture extends through the soft tissue, disposition of the bridge post anchor in bone can secure the soft tissue to the bone;

inserting the center post anchor into the bone;

passing the second portion of the suture through the soft tissue;

cinching the suture so as to draw the soft tissue against the bone; and

securing the second portion of the suture to the bone by inserting the bridge post anchor into the bone, with the capture element capturing the second portion of the suture to the bone.

In another form of the present invention, there is provided a system for attaching soft tissue to bone, the system comprising:

a center post anchor comprising a body having a distal end and a proximal end, a pair of legs extending distally from the distal end of the body and separated by a slot, the legs tapering outwardly along their length so that the center post anchor has a diameter at the legs which is larger than the diameter at the body, with the legs being inwardly compressible, and a suture attached to the proximal end of the body, the suture having at least one free end associated therewith; and

at least one bridge post anchor comprising a body having a distal end and a proximal end, and a pair of legs extending from the distal end of the body and separated by a slot, the legs tapering outwardly along their length so that the bridge post anchor has a diameter at the legs which is larger than the diameter at the body, with the legs being inwardly compressible;

such that the center post anchor can be secured in a hole in a bone by compressing its legs inwardly, deploying the center post anchor in the bone and releasing its legs so that they thereafter engage the bone, whereby to secure the suture to the bone;

and further such that the bridge post anchor can capture the free end of the suture to bone by positioning the free end of the suture in the slot, compressing the legs of the bridge post anchor inwardly, positioning the bridge post anchor in a hole in the bone, and releasing its legs so that they thereafter engage the bone.

### BRIEF DESCRIPTION OF THE DRAWINGS

These and other objects and features of the present invention will be more fully disclosed or rendered obvious by the following detailed description of the invention, which is to be considered together with the accompanying drawings wherein like numbers refer to like parts, and further wherein:

FIGS. 1A-1D are schematic views showing various aspects of hip motion;

FIG. 2 is a schematic view showing the bone structure in the region of the hip joints;

FIG. 3 is a schematic view of the femur;

FIG. 4 is a schematic view of the top end of the femur;

FIG. 5 is a schematic view of the pelvis;

FIGS. 6-12 are schematic views showing the bone and soft tissue structure of the hip joint;

FIG. 13 is a schematic view showing cam-type femoroacetabular impingement (FAI);

FIG. 14 is a schematic view showing pincer-type femoroacetabular impingement (FAI);

FIG. 15 is a schematic view showing a labral tear;

FIG. 16 is a schematic view showing the labrum attached to the acetabulum;

FIG. 17 is a schematic view showing the labrum detached from the acetabulum;

FIG. 18 is a schematic view showing a novel anchoring system formed in accordance with the present invention, wherein the anchoring system comprises a center post anchor and a pair of bridge post anchors;

FIG. 19 is a schematic view showing further details of the center post anchor of FIG. 18;

FIG. 20 is a schematic view showing further details of a bridge post anchor of FIG. 18;

FIGS. 21-23 are schematic views showing the anchoring system of FIG. 18 being used to re-attach the labrum to the acetabulum;

FIG. 23A is a schematic view showing another center post anchor formed in accordance with the present invention;

FIG. 23B is a schematic view showing another bridge post anchor formed in accordance with the present invention;

FIGS. 24 and 25 are schematic views showing still another bridge post anchor formed in accordance with the present invention;

FIGS. 26-29 are schematic views showing operation of the bridge post anchor of FIGS. 24 and 25;

FIGS. 29A and 29B show alternative constructions for the bridge post anchor; and

FIG. 30 is a schematic view showing yet another bridge post anchor formed in accordance with the present invention.

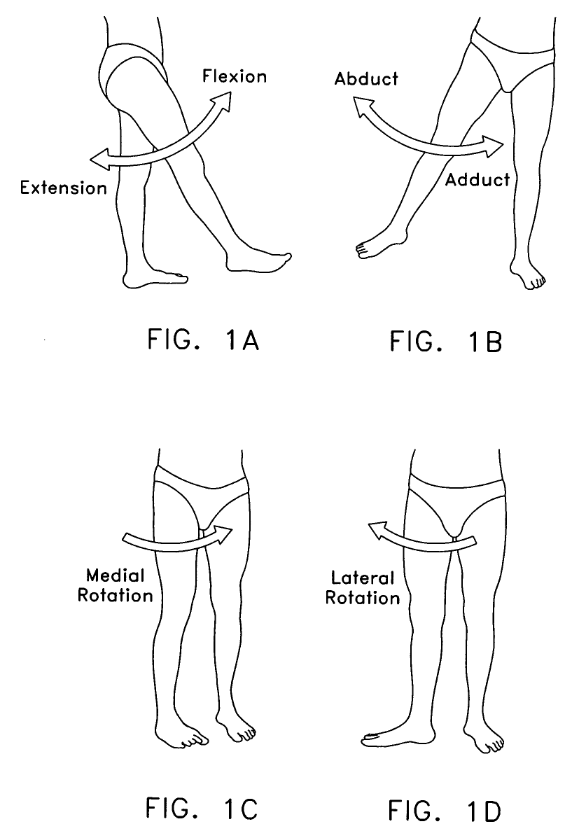

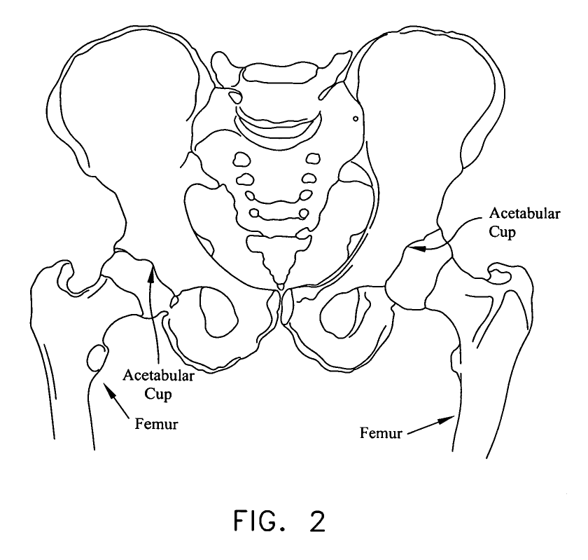

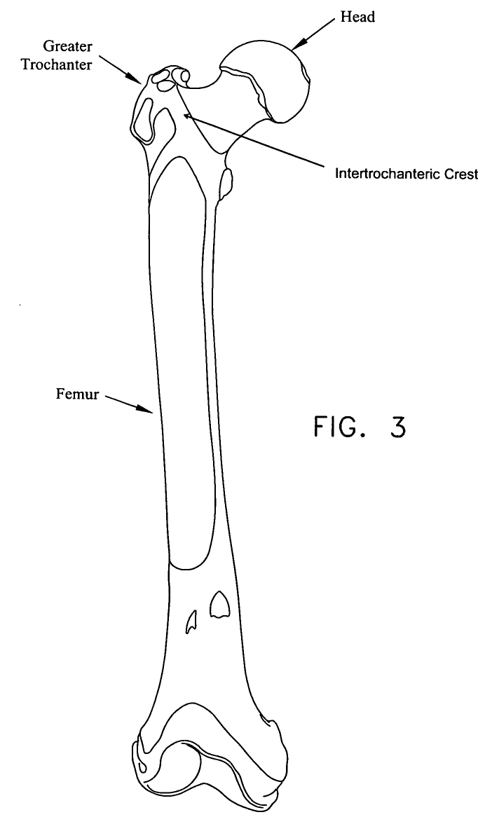

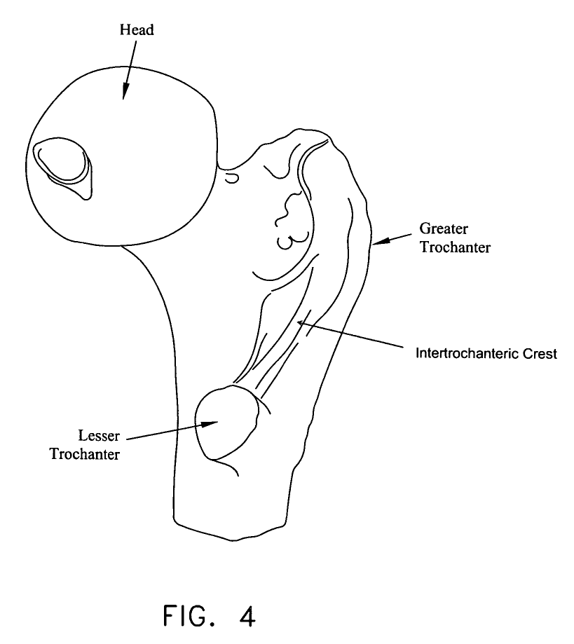

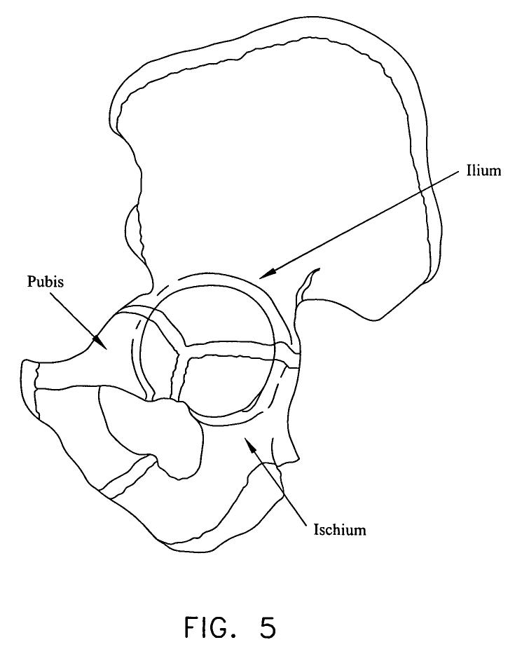

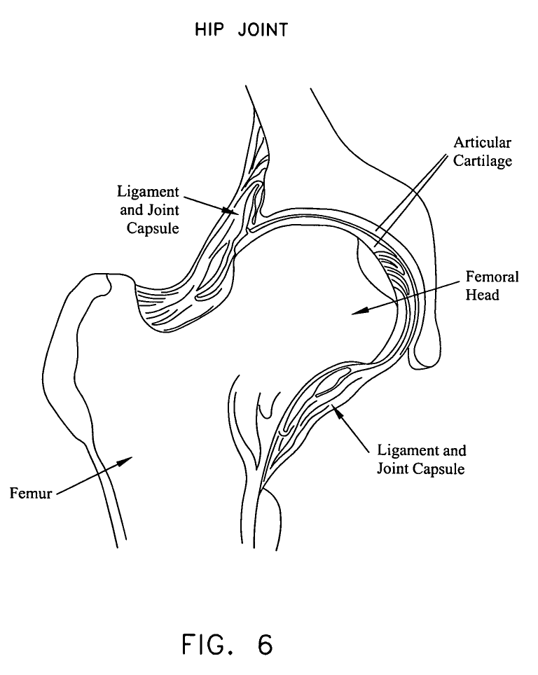

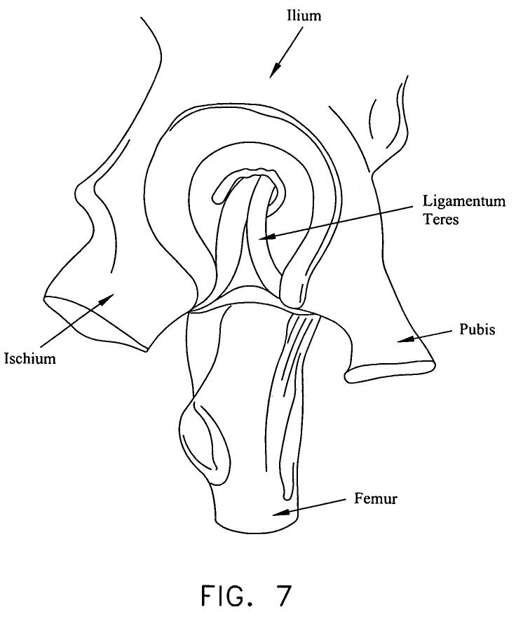

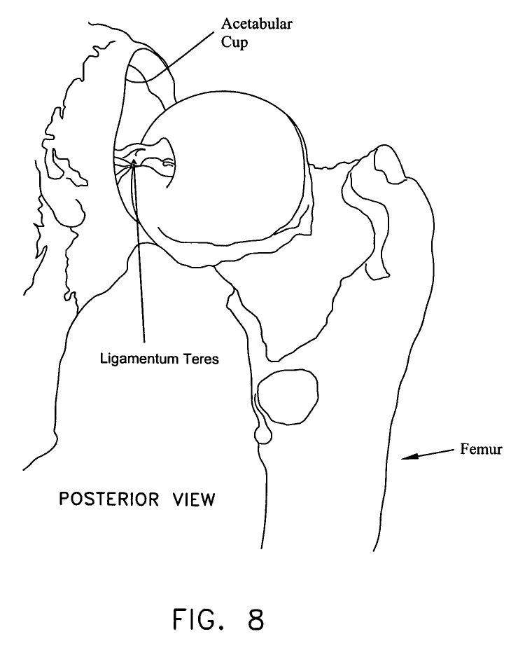

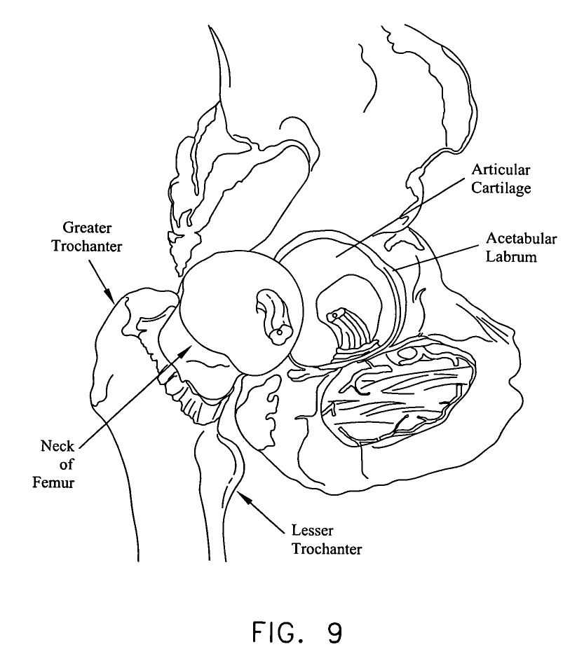

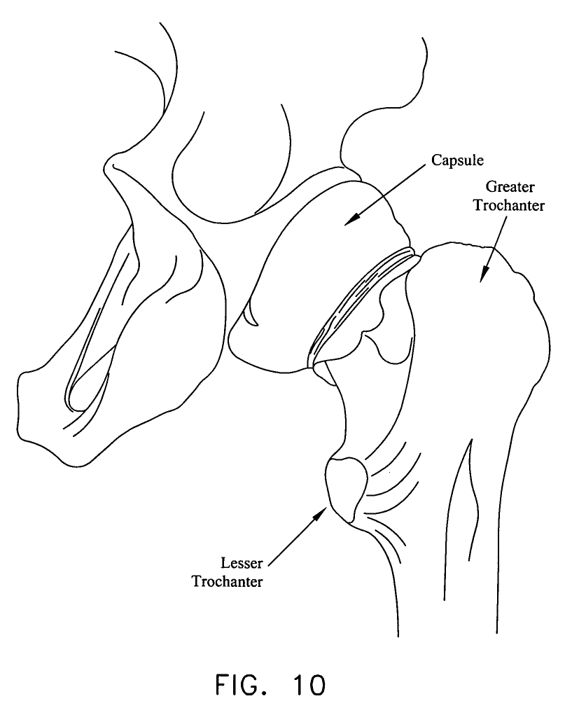

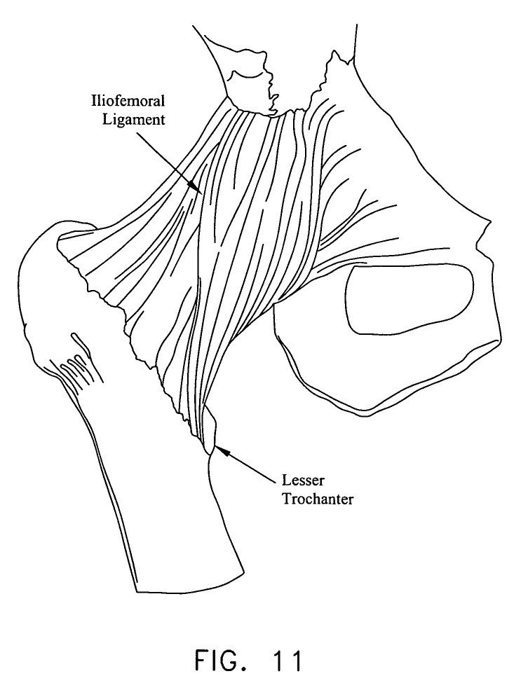

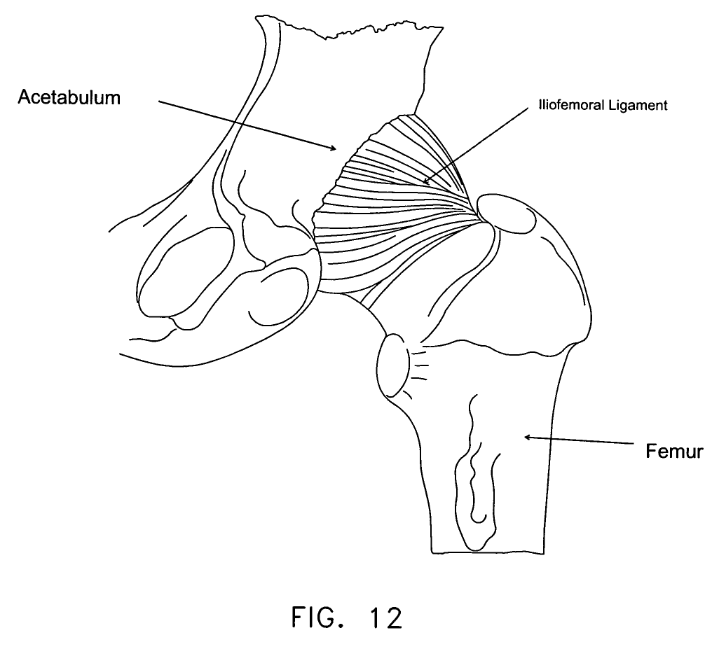

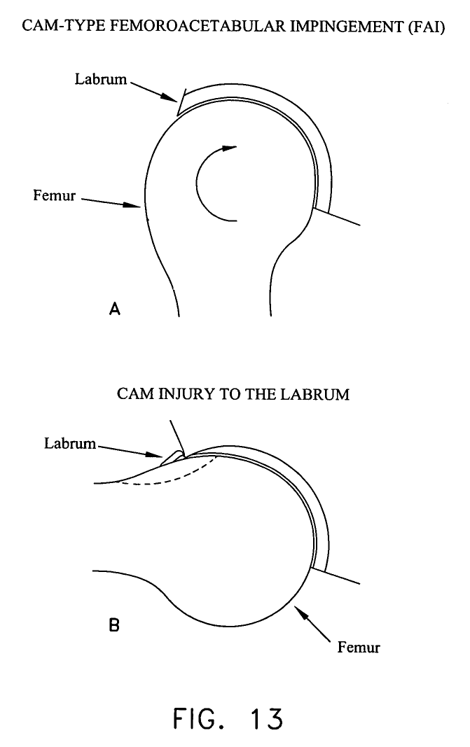

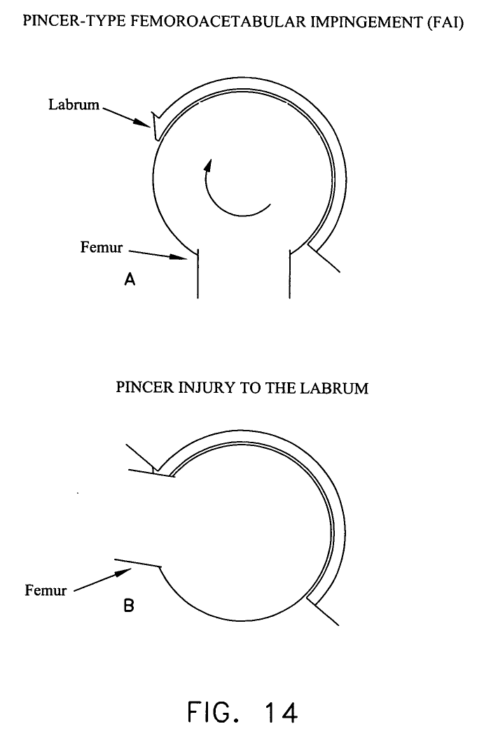

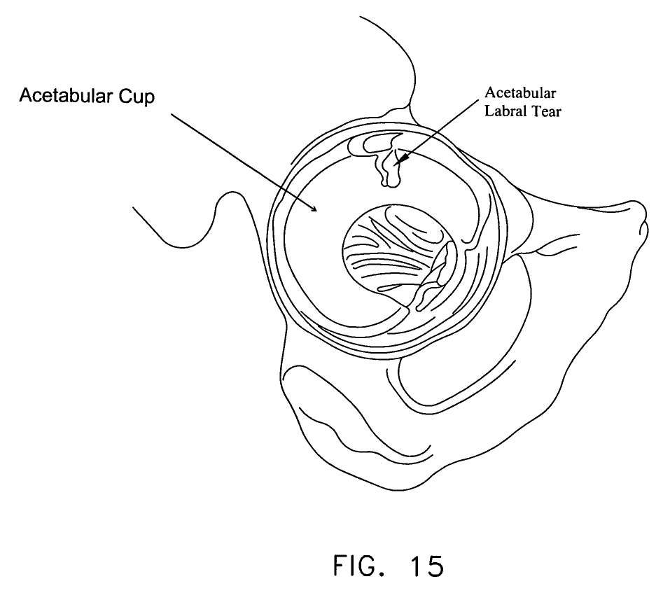

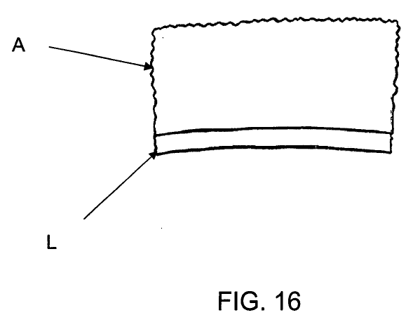

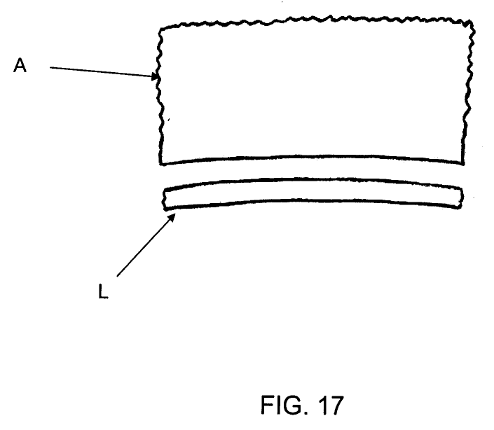

### DETAILED DESCRIPTION OF THE INVENTION

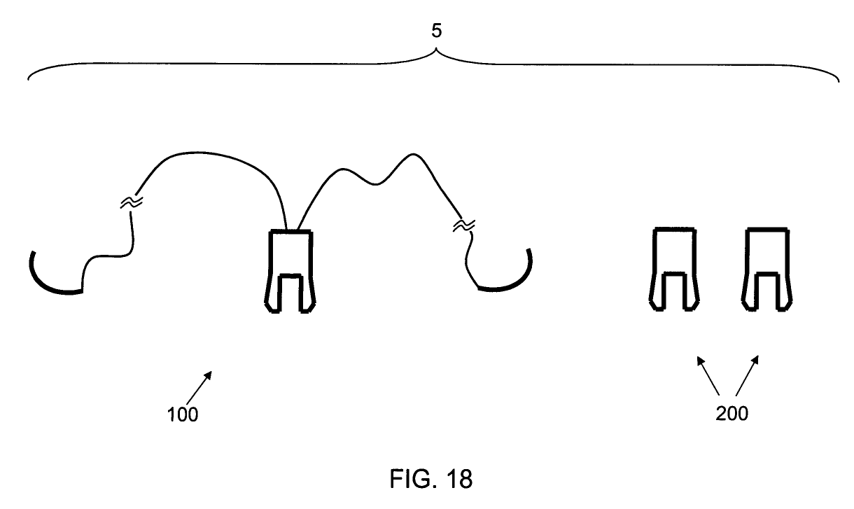

Looking now at FIG. 18, there is shown novel anchoring system 5 for re-attaching the labrum to the acetabulum. This novel anchoring system 5 generally comprises a center post anchor 100 and a pair of bridge post anchors 200.

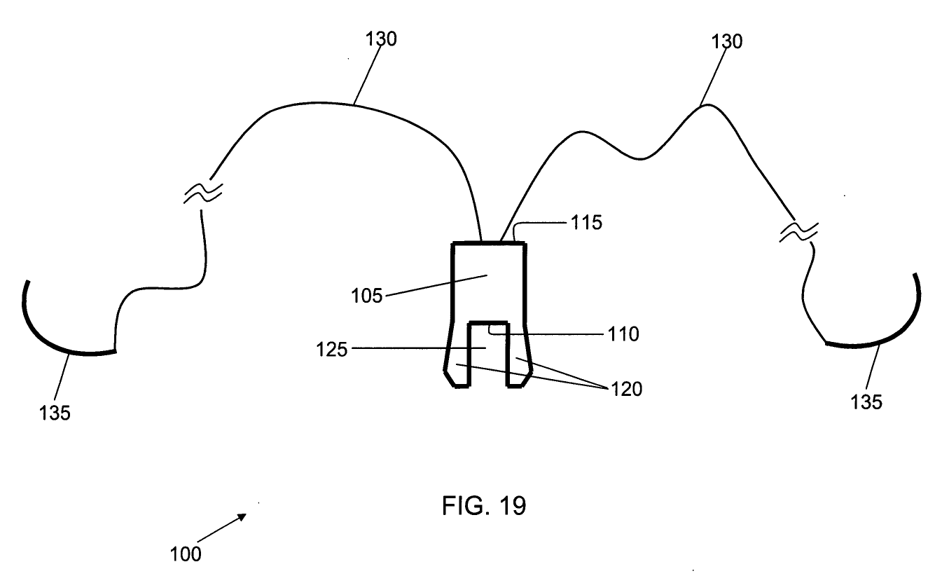

Looking next at FIG. 19, center post anchor 100 generally comprises a body 105 having a distal end 110 and a proximal end 115. A pair of legs 120 extend distally from distal end 110 of body 105. Legs 120 are separated by a slot 125. Legs 120 taper outwardly along their length, so that center post anchor 100 has a diameter at legs 120 which is somewhat larger than the diameter of body 105. Legs 120 have a selected degree of resiliency, such that the distal ends of legs 120 can be compressed inboard when desired, so that legs 120 can have a combined diameter equal to or less than body 105 of center post anchor 100. A pair of sutures 130 are secured to proximal end 115 of body 105. Each of sutures 130 has a needle 135 secured to its free end.

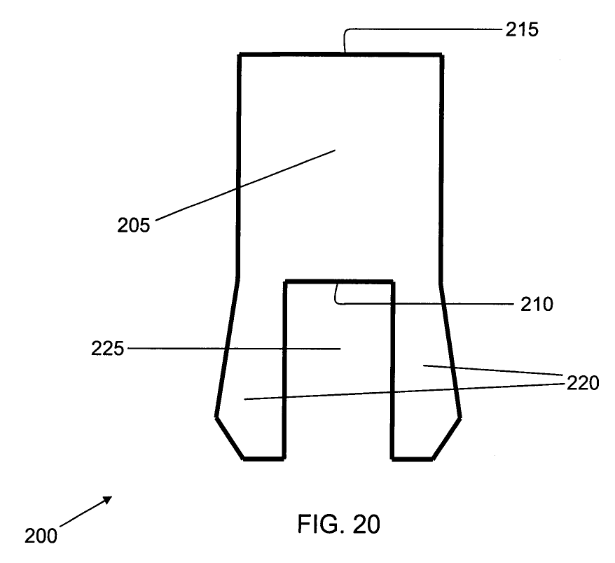

Looking next at FIG. 20, each bridge post anchor 200 is preferably identical to center post anchor 100, except that it does not have sutures 130 and needles 135 secured thereto. More particularly, each bridge post anchor 200 generally comprises a body 205 having a distal end 210 and a proximal end 215. A pair of legs 220 extend distally from distal end 210 of body 205. Legs 220 are separated by a slot 225. Legs 220 taper outwardly along their length, so that bridge post anchor 200 has a diameter at legs 220 which is somewhat larger than the diameter of body 205. Legs 220 have a selected degree of resiliency, such that the distal ends of legs 220 can be compressed inboard when desired, so that legs 220 can have a combined diameter equal to or less than body 205 of bridge post anchor 200.

### Use of Anchoring System 5 to Re-Attach the Labrum to the Acetabulum

Anchoring system 5 is preferably used as follows to re-attach the labrum to the acetabulum.

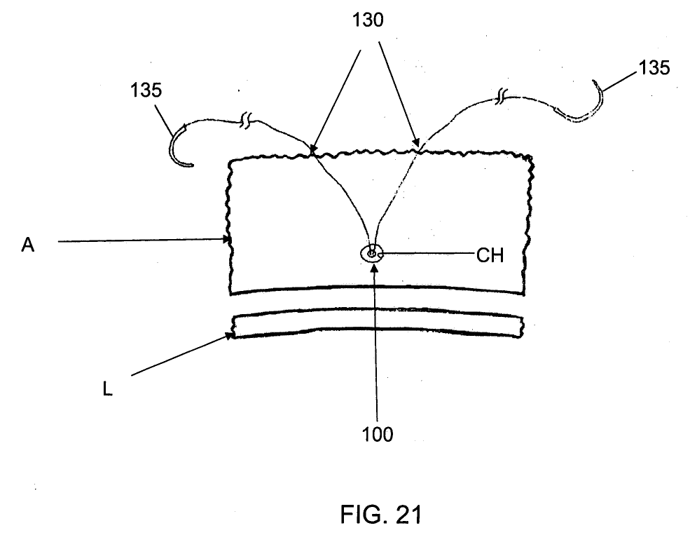

Looking now at FIG. 21, a center hole CH is formed in acetabulum A adjacent to the acetabular rim. Then center post anchor 100 is secured in center hole CH. This is done by (i) compressing legs 120 inboard so that legs 120 have a combined diameter equal to or less than body 105 of center post anchor 100, (ii) inserting center post anchor 100 into center hole CH while its legs are in this inboard position, and (iii) releasing legs 120, thereby causing legs 120 to engage the side wall of center hole CH and thereby secure center post anchor 100 to acetabulum A. At the same time, sutures 130 extend from proximal end 115 of center post anchor 100.

Preferably, the distal ends of legs 120 are beveled inwardly at their peripheries so that engagement of legs 120 with the rim of center hole CH during anchor insertion automatically causes legs 120 to compress inwardly to facilitate entry into center hole CH and thereafter automatically project outwardly so as to grip the surrounding bone.

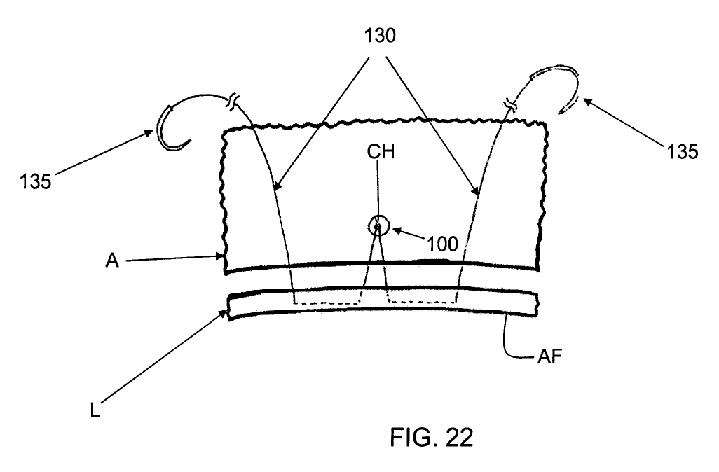

Looking next at FIG. 22, needles 135 are then used to pass sutures 130 through labrum L. Preferably, each of the sutures 130 is passed through labrum L so that sutures 130 extend out of labrum L laterally from where sutures 130 enter the labrum. As a result, sutures 130 do not project through the articular face AF of labrum L.

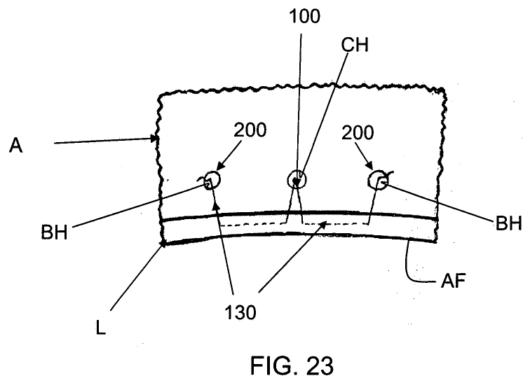

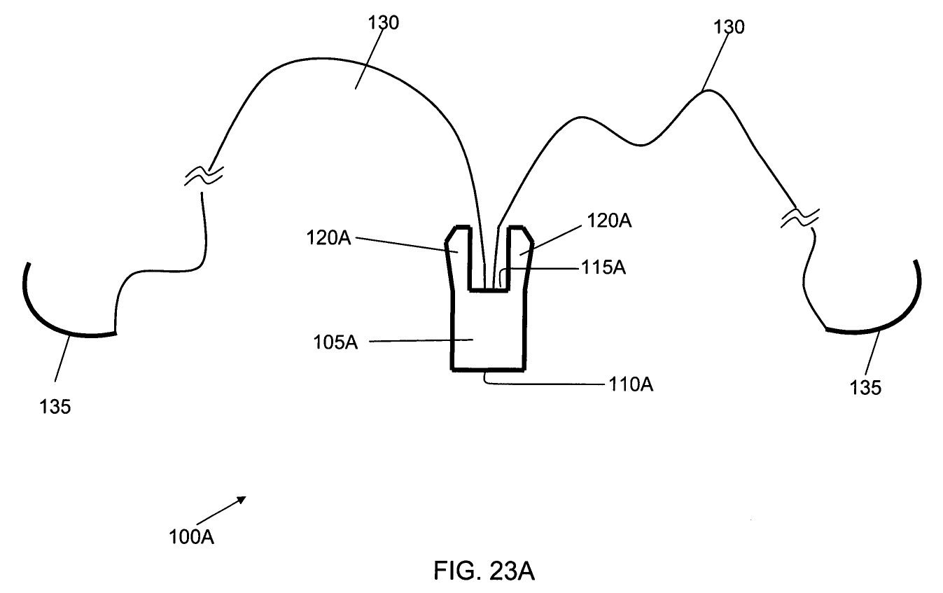

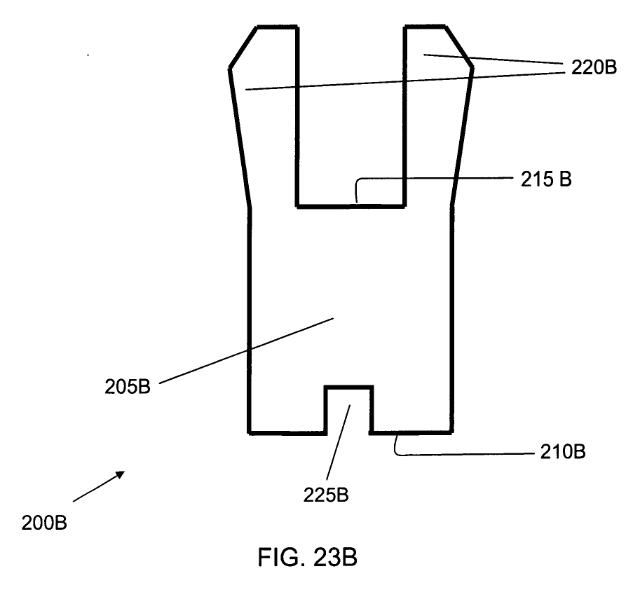

Thereafter, and looking now at FIG. 23, a pair of bridge holes BH are formed in acetabulum A, one on each side of center post anchor 100. Then bridge post anchors 200 are used to secure the free ends of sutures 130 to acetabulum A so as to re-attach labrum L to acetabulum A. More particularly, while holding a suture 130 taut, so as to draw labrum L against acetabulum A, bridge post anchor 200 is moved so as to straddle that suture, with the suture being positioned in anchor slot 225, against distal end 210 of body 205. Still holding the suture taut, legs 220 of bridge post anchor 200 are compressed so that legs 220 have a combined diameter equal to or less than body 205 of bridge post anchor 200, and then bridge post anchor 200 is pressed into bridge hole BH, with distal end 210 of body 205 carrying suture 130 into bridge hole BH. Thereafter, the compression on legs 220 is released whereupon legs 220 engage the side wall of bridge hole BH so as to secure bridge post anchor 200 to acetabulum A. This action has the effect of binding suture 130 to acetabulum A under tension, thereby fixing labrum L to acetabulum A.

Preferably, the distal ends of legs 220 are beveled inwardly at their peripheries so that engagement of legs 220 with the rim of bridge hole BH during anchor insertion automatically causes legs 220 to compress inwardly to facilitate entry into bridge hole BH and thereafter automatically project outwardly so as to grip the surrounding bone.

This procedure is then repeated for the remaining suture 130, i.e., passing suture 130 through labrum L and then knotlessly attaching that suture under tension to acetabulum A using a bridge post anchor 200 so as to secure labrum L to the acetabulum. Then the free ends of the sutures extending out of bridge holes BH (including needles 135) are cut away, and the suture ends and needles are removed from the surgical site.

It will be appreciated that the foregoing labrum re-attachment is effected without the need to tie a knot. As a result, the present invention provides a simpler, faster and more convenient approach for securing the labrum to the acetabulum.

It should also be appreciated that if it is desired to use only one suture to secure the labrum to the acetabulum, only one of the sutures 130 and needle 135, and only one bridge post anchor 200, is used. In this case, the unused suture 130 and needle 135 may be cut away, adjacent to center post anchor 100.

Alternatively, where it is desired to use only one suture strand to secure the labrum to the acetabulum, anchoring system 5 may be provided with a center post anchor 100 and only one bridge post anchor 200. In this construction, center post anchor 100 may be provided with only one suture strand 130 extending therefrom.

It should also be appreciated that, if desired, an arthroscopic suture passer can be used in place of needles 135 to pass each of sutures 130 through labrum L. By way of example but not limitation, the suture passers described in U.S. Pat. Nos. 5,522,820 and Des. 343,728, and the suture passers described in U.S. Patent Application Publications Nos. 2005/0283171 and 2007/0179510, may be used to pass each of the sutures 130 through labrum L. Where an arthroscopic suture passer is to be used in place of needles 135 to pass suture 130 through labrum L, needles 135 are omitted.

### Use of a Different Center Post Anchor

It should also be appreciated that center post anchor 100 may be replaced by another device for anchoring suture to bone.

By way of example but not limitation, center post anchor 100 may be replaced by a conventional screw-type bone anchor of the sort sold by Depuy Mitek under the trade name SPIRALOK, or a conventional barb-type bone anchor of the sort sold by Depuy Mitek under the trade name GII QUICKANCHOR, or a conventional toggle-type bone anchor of the sort sold by Depuy Mitek under the trade name PANALOK, etc.

By way of example but not limitation, and looking now at FIG. 23A, there is shown a center post anchor 100A which may be used in place of center post anchor 100 described previously.

More particularly, each center post anchor 100A generally comprises a body 105A having a distal end 110A and a proximal end 115A. A pair of legs 120A extend proximally from proximal end 115A of body 105A. Legs 120A taper outwardly along their length, so that center post anchor 100A has a diameter at legs 120A which is somewhat larger than the diameter of body 105A. Legs 120A have a selected degree of resiliency, such that the proximal ends of legs 120A can be compressed inboard when desired, so that legs 120A can have a combined diameter equal to or less than body 105A of center post anchor 100A. Sutures 130 are secured to proximal end 115A of body 105A. In use, center post anchor 100A is driven distal end first into center hole CH, with legs 120A first compressing inboard so as to enter the acetabulum and thereafter expanding outboard so as to secure center post anchor 100A center hole CH.

### Use of a Different Bridge Post Anchor

It should also be appreciated that bridge post anchor 200 may be replaced by another device for anchoring suture to bone.

By way of example but not limitation, and looking now at FIG. 23B, there is shown a bridge post anchor 200B which may be used in place of bridge post anchor 200 described previously.

More particularly, each bridge post anchor 200B generally comprises a body 205B having a distal end 210B and a proximal end 215B. A pair of legs 220B extend proximally from proximal end 215B of body 205B. Legs 220B taper outwardly along their length, so that bridge post anchor 200B has a diameter at legs 220B which is somewhat larger than the diameter of body 205B. Legs 220B have a selected degree of resiliency, such that the proximal ends of legs 220B can be compressed inboard when desired, so that legs 220B can have a combined diameter equal to or less than body 205B of bridge post anchor 200B. A slot 225B is formed on distal end 210B of body 205B. In use, suture 130 is engaged in slot 225B of body 205B, and then bridge post anchor 200B is driven distal end first into bridge hole BH, with legs 220B first compressing inboard so as to enter the acetabulum and thereafter expanding outboard so as to secure bridge post anchor 200B bridge hole BH.

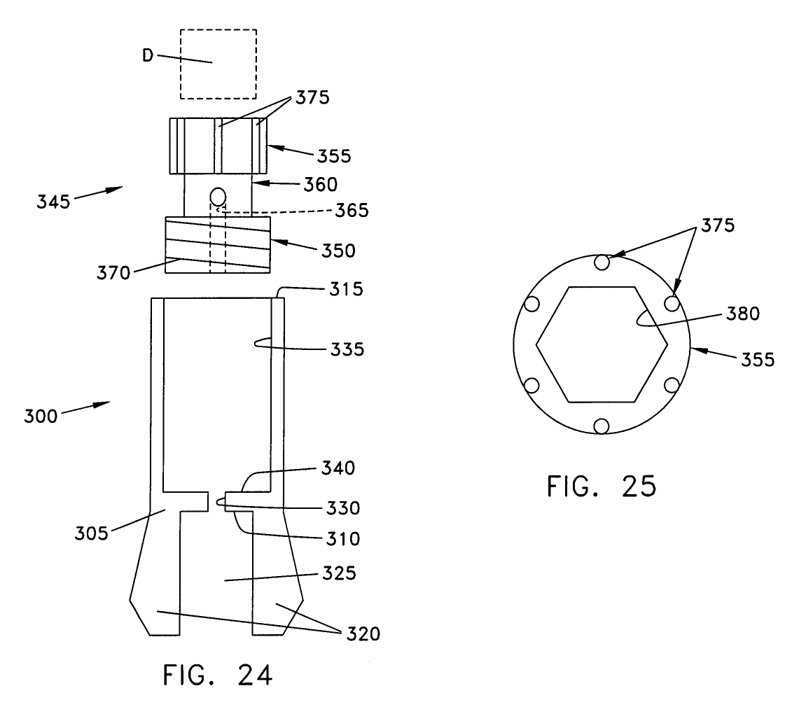

By way of further example but not limitation, and looking now at FIGS. 24 and 25, there is shown a bridge post anchor 300 which can be used to anchor suture 130 to the acetabulum. Bridge post anchor 300 generally comprises a body 305 having a distal end 310 and a proximal end 315. A pair of legs 320 extend distally from distal end 310 of body 305. Legs 320 are separated by a slot 325. Legs 320 taper outwardly along their length, so that bridge post anchor 300 has a diameter at legs 320 which is somewhat larger than the diameter of body 305. Legs 320 have a selected degree of resiliency, such that the distal ends of legs 320 can be compressed inboard when desired, so that legs 320 can have a combined diameter equal to or less than body 305 of bridge post anchor 300.

Bridge post anchor 300 also comprises a bore 330 which opens on distal end 310 of body 305, intermediate legs 320. Bore 330 extends proximally and intersects a threaded counterbore 335. Bore 330 and counterbore 335 define an annular shoulder 340 at their intersection.

Bridge post anchor 300 also comprises a suture spool 345 which is adapted to be movably received within threaded counterbore 335 of body 305. More particularly, suture spool 345 comprises a distal hub 350, a proximal hub 355, and a neck 360 extending therebetween. A passageway 365 opens on the distal end of distal hub 350 and extends proximally so as to open on the outer surface of neck 360. A screw thread 370 is formed on distal hub 350. Screw thread 370 is sized to engage the threaded counterbore 335 in body 305. A plurality of bores 375 extend through proximal hub 355. A non-circular (e.g., hexagonal) opening 380 is formed in proximal hub 355. Non-circular opening 380 receives a conventional rotary driver (e.g., a hex driver) D. Rotary driver D may be used to turn suture spool 345, so as to move suture spool 345 within counterbore 335 and hence relative to body 305.

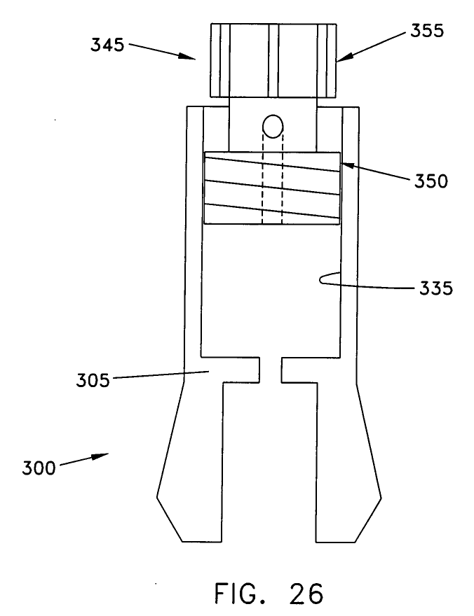

Prior to use, bridge post anchor 300 is configured so that suture spool 345 has its distal hub 350 screwed into counterbore 335 of body 305, with proximal hub 355 extending out of body 305 (see FIG. 26).

Bridge post anchor 300 is preferably used as follows to re-attach the labrum to the acetabulum.

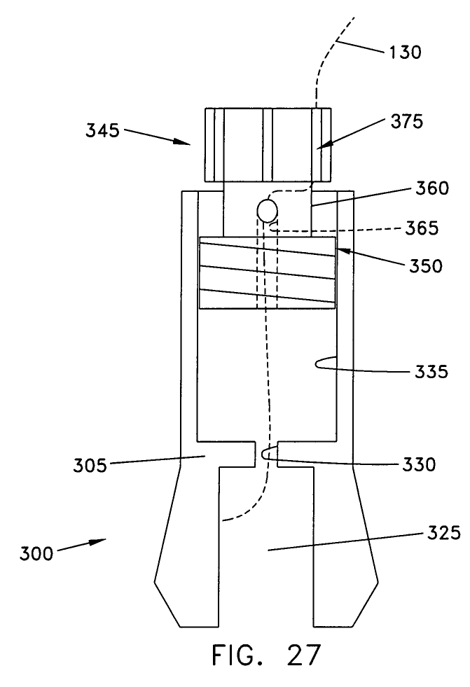

After center post anchor 100 has been positioned in the acetabulum and a suture 130 has been passed through the labrum, the free end of suture 130 is passed outside the body and then it is threaded through bridge post anchor 300, which also resides outside the body (FIG. 27). More particularly, the free end of suture 130 is threaded through bridge post anchor 300 by passing the free end of the suture through slot 325 of body 305, through bore 330 of body 305, through passageway 365 of suture spool 345, along the outside of neck 360 of suture spool 345, and then through one of the plurality of bores 375 extending through proximal hub 355, so that a free end of suture 130 extends from proximal hub 355 of suture spool 345. It should be appreciated that as the free end of suture 130 extends through anchor body 305 and suture spool 345, the free end of the suture 130 follows a tortuous path which provides some, but not complete, resistance to suture movement relative to anchor body 305 and suture spool 345.

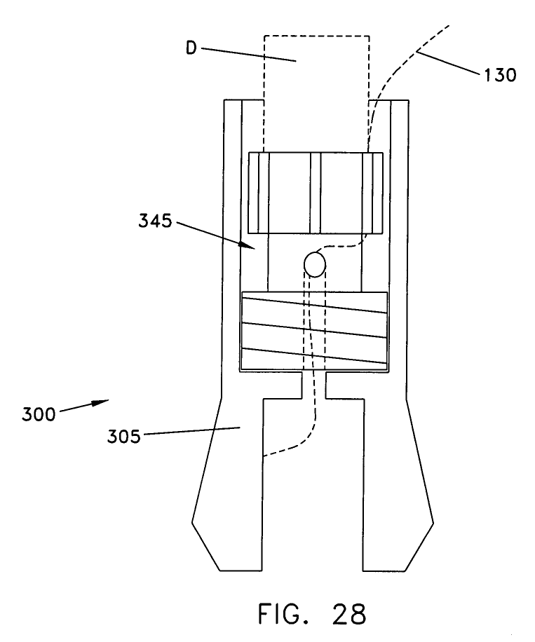

Then, using driver D, suture spool 345 is advanced down body 305 of bridge post anchor 300 (FIG. 28).

Next, bridge post anchor 300 is advanced down the free end of suture 130 so that it enters the patient and is delivered to the surgical site. As this occurs, there is some resistance to distal motion of bridge post anchor 300 on the suture, due to the tortuous path followed by suture 130 through bridge post anchor 300, however, this may be overcome by applying steady distal force to the bridge post anchor.

Bridge post anchor 300 is brought adjacent to a bridge hole BH formed in acetabulum A. Then, with the free end of suture 130 being pulled slightly proximally so as to take up slack, legs 320 of bridge post anchor 300 are compressed and bridge post anchor 300 is pressed into bridge hole BH. Again, as this occurs, there is some resistance to distal motion of bridge post anchor 300 on the suture, due to the tortuous path followed by suture 130 through bridge post anchor 300. Thereafter, the compression on legs 320 is released, whereupon legs 320 engage the side wall of bridge hole BH so as to secure bridge post anchor 300 to acetabulum A.

Again, the distal ends of legs 320 are preferably beveled inwardly at their peripheries so that engagement of legs 320 with the rim of bridge hole BH during anchor insertion automatically causes legs 320 to compress inwardly to facilitate entry into bridge hole BH and thereafter automatically project outwardly so as to grip the surrounding bone.

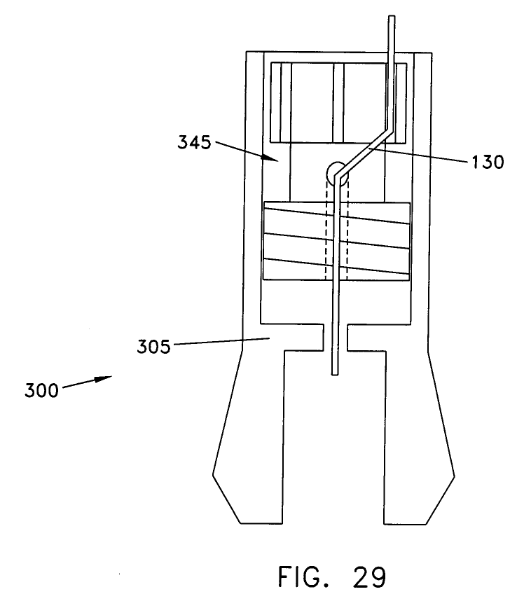

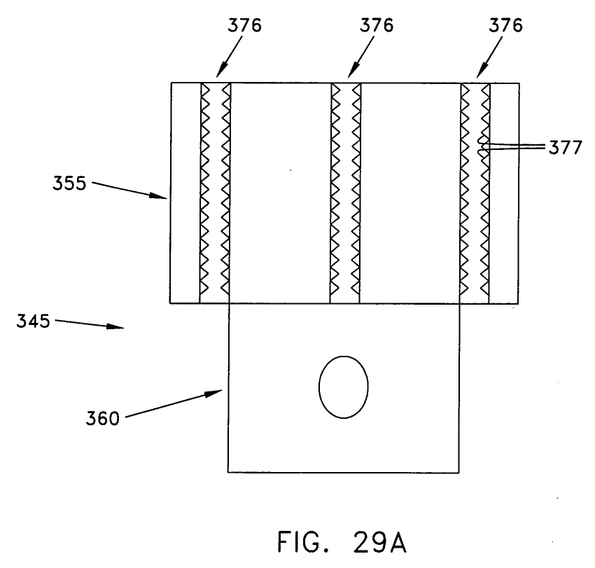

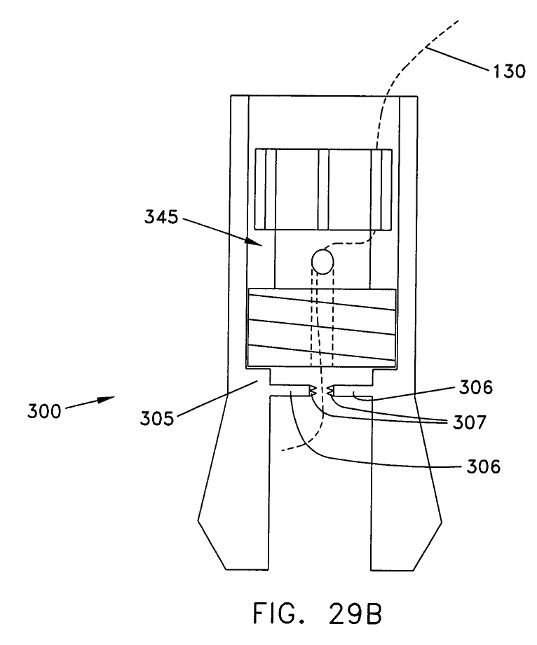

Then, with suture 130 held under substantial tension, driver D (engaged in non-circular hole 380) is used to retract suture spool 345 proximally within body 305 (FIG. 29). As this occurs, the substantial tension on the free end of suture 130 causes the suture to bind on suture spool 345, due to the tortuous path followed by suture 130 through the suture spool. As a result, proximal motion of suture spool 345 relative to body 305 (and hence proximal motion of suture spool 345 relative to acetabuum A, within which body 305 is fixed) has the effect of cinching the free end of suture 130. Inasmuch as suture 130 is threaded through labrum L, this action has the effect of drawing the labrum to the acetabulum, so as to effect the desired labral re-attachment.

Once cinching is complete, the procedure is then repeated using another bridge post anchor 300 to secure the remaining suture 130. Then the free ends of the sutures extending out of bridge holes BH are cut away, and the sutures are removed from the surgical site.

As discussed above, suture 130 is intended to be threaded through bridge post anchor 300 by passing the free end of the suture through slot 325 of body 305, through bore 330 of body 305, through passageway 365 of suture spool 345, along the outside of neck 360 of suture spool 345, and then through one of the plurality of bores 375 extending through proximal hub 355, so that a free end of suture 130 extends from proximal hub 355 of suture spool 345. This approach results in the free end of suture 130 following a tortuous path through the bridge post anchor. This tortuous path provides some, but not complete, resistance to suture movement relative to body 305 and suture spool 345, such that longitudinal movement of suture spool 345 relative to body 305 can effect the aforementioned suture cinching.

Alternatively, if desired, and looking now at FIG. 29A, bores 375 in proximal hub 355 of suture spool 345 can be replaced with slots 376 having teeth 377 therein. Slots 376 and teeth 377 essentially form a suture cleat for securing the free end of suture 130 to suture spool 345. More particularly, when the free end of suture 130 is to be secured to suture spool 345, the free end of the suture is slipped into one of the slots 376, where it is gripped by teeth 377. As a result, subsequent movement of suture spool 345 within body 305 carries suture 130 with it, whereby to permit suture cinching as discussed above.

Alternatively, or in addition to the foregoing, it is also possible to modify body 305 of bridge post anchor 300 so as to provide a cam-type cleat within body 305 of the anchor. More particularly, and looking now at FIG. 29B, body 305 can be formed so that the walls 306 defining bore 330 are substantially resilient in the proximal direction but substantially rigid in the distal direction, and bore 330 can be formed with opposing teeth 307. As a result of this construction, when the free end of suture 130 is advanced through bore 330 and is thereafter pulled proximally, walls 306 can yield proximally so as to permit suture 130 to pass thereby. However, since walls 306 are unable to yield in the distal direction, suture 130 is unable to return distally. Thus, walls 306 and teeth 307 effectively form a cam-type cleat within body 305 of the bridge post anchor, such that subsequent movement of suture spool 345 within body 305 carries suture 130 with it, whereby to permit suture cinching as discussed above. Of course, with this design, it is important that suture spool 345 sit sufficiently proximally of walls 306 to permit walls 306 to yield proximally as just discussed.

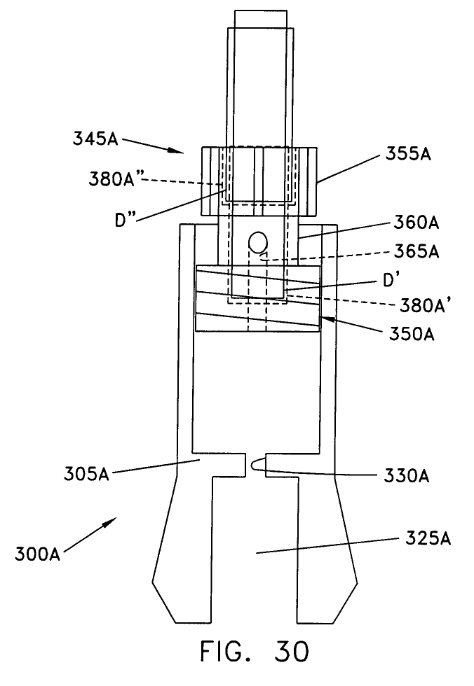

Alternatively, and looking now at FIG. 30, a bridge post anchor 300A can be provided which is generally similar to bridge post anchor 300 described above, but which utilizes a suture spool 345A which permits the suture to be wound around neck 360A of suture spool 345A. More particularly, in this form of the invention, suture spool 345A is constructed so that its proximal hub 355A can rotate about distal hub 350A and neck 360A so as to wrap suture 130 around the neck, thereby increasing stability of suture position upon completion of the labrum attachment. In this form of the invention, distal hub 350A and neck 360A may comprise a non-circular opening 380A′ for turning the distal hub and neck as a unit, and proximal hub 355A may comprise a non-circular opening 380A″ for turning proximal hub 355A. Driver D may comprise coaxial elements D′ and D″, with element D′ turning distal hub 350A and neck 360A as a unit, and with element D″ turning proximal hub 355A.

If desired, the aforementioned suture cleats in proximal hub 355 of suture spool 345 (FIG. 29A) and/or the cam-type cleat within body 305 of the bridge post anchor (FIG. 29B) can be combined with the construction where proximal hub 355 is turned separately from distal hub 350 and neck 360 (FIG. 30).

Furthermore, still other constructions will be apparent to those skilled in the art whereby suture spool 345 may be turned independently of body 305 so as to cinch the suture holding labrum L.

### Trans-Labral Anchor Fixation

In the foregoing description, the bodies of center post anchor 100 and distal post anchors 200, 300 are described as being deployed directly into acetabulum A, without first passing through labrum L. However, it should be appreciated that, if desired, the bodies of center post anchor 100 and/or distal post anchors 200, 300 may be deployed in acetabulum A trans-labrally, i.e., by passing through the labrum before entering the acetabulum. This approach can eliminate the additional step of passing the suture through the labrum after the center post anchor has been set, and can obviate the need for an independent suture passing device (e.g., needle 135 or an independent suture passer instrument such as disclosed above).

### Use of the Novel Method and Apparatus for Other Joints, Etc.

It should be appreciated that the novel method and apparatus of the present invention may be used for attaching other tissues and the like to the acetabulum, and/or may be used for attaching other tissues and the like to other bones. By way of example but not limitation, the novel method and apparatus of the present invention may be used to attach soft tissue and prostheses in the knee joint, in the shoulder joint, etc.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A system for securing soft tissue to bone, the system comprising:
a center post anchor comprising a body adapted for disposition in bone and having a retention element thereon for retaining the body in bone, the center post anchor comprising a suture having a first portion secured to the body and a second portion residing free of the body and adapted to be passed through the soft tissue which is to be secured to the bone; and
a bridge post anchor comprising a body adapted for disposition in bone and having a retention element thereon for retaining the body in bone, the bridge post anchor including a capture element for capturing the second portion of the suture to the bone, such that when the center post anchor is disposed in bone and the second portion of the suture extends through the soft tissue, disposition of the bridge post anchor in bone can secure the soft tissue to the bone.

2. A system according to claim 1 wherein the soft tissue comprises the labrum.

3. A system according to claim 1 wherein the bone comprises the acetabulum.

4. A system according to claim 1 wherein the retention element of the center post anchor comprises a pair of legs extending from the body, wherein the legs taper outwardly along their length so that the center post anchor has a diameter at the legs which is larger than the diameter of the body, and further wherein the legs are inwardly compressible.

5. A system according to claim 4 wherein the legs of the center post anchor extend distally from the body.

6. A system according to claim 4 wherein the legs of the center post anchor extend proximally from the body.

7. A system according to claim 1 wherein the retention element of the bridge post anchor comprises a pair of legs extending from the body, wherein the legs taper outwardly along their length so that the bridge post anchor has a diameter at the legs which is larger than the diameter of the body, and further wherein the legs are inwardly compressible.

8. A system according to claim 7 wherein the legs of the bridge post anchor extend distally from the body.

9. A system according to claim 7 wherein the legs of the bridge post anchor extend proximally from the body.

10. A system according to claim 1 wherein the capture element of the bridge post anchor comprises a slot formed in the bridge post anchor.

11. A system according to claim 1 wherein the second portion of the suture has a needle attached thereto.

12. A system according to claim 1 wherein the suture comprises a third portion secured to the body and a fourth portion residing free of the body and adapted to be passed soft tissue which is to be secured to the bone, and further wherein the system further comprises a second bridge post anchor for securing the fourth portion of the suture to the bone.

13. A system according to claim 1 wherein the capture element of the bridge post anchor comprises a suture spool movably mounted to the body of the bridge post anchor.

14. A system according to claim 13 wherein the suture spool is rotatable so as to cinch the suture.

15. A system according to claim 13 wherein the suture spool is longitudinally movable relative to the body so as to cinch the suture.

16. A method for securing soft tissue to bone, the method comprising:
providing a system comprising:
a center post anchor comprising a body adapted for disposition in bone and having a retention element thereon for retaining the body in bone, the center post anchor comprising a suture having a first portion secured to the body and a second portion residing free of the body and adapted to be passed through the soft tissue which is to be secured to the bone; and
a bridge post anchor comprising a body adapted for disposition in bone and having a retention element thereon for retaining the body in bone, the bridge post anchor including a capture element for capturing the second portion of the suture to the bone, such that when the center post anchor is disposed in bone and the second portion of the suture extends through the soft tissue, disposition of the bridge post anchor in bone can secure the soft tissue to the bone;
inserting the center post anchor into the bone;
passing the second portion of the suture through the soft tissue;
cinching the suture so as to draw the soft tissue against the bone; and
securing the second portion of the suture to the bone by inserting the bridge post anchor into the bone, with the capture element capturing the second portion of the suture to the bone.

17. A method according to claim 16 wherein the soft tissue comprises the labrum.

18. A method according to claim 16 wherein the bone comprises the acetabulum.

19. A method according to claim 16 wherein the retention element of the center post anchor comprises a pair of legs extending from the body, wherein the legs taper outwardly along their length so that the center post anchor has a diameter at the legs which is larger than the diameter of the body, and further wherein the legs are inwardly compressible.

20. A method according to claim 19 wherein the legs of the center post anchor extend distally from the body.

21. A method according to claim 19 wherein the legs of the center post anchor extend proximally from the body.

22. A method according to claim 16 wherein the retention element of the bridge post anchor comprises a pair of legs extending from the body, wherein the legs taper outwardly along their length so that the bridge post anchor has a diameter at the legs which is larger than the diameter of the body, and further wherein the legs are inwardly compressible.

23. A method according to claim 22 wherein the legs of the bridge post anchor extend distally from the body.

24. A method according to claim 22 wherein the legs of the bridge post anchor extend proximally from the body.

25. A method according to claim 16 wherein the capture element of the bridge post anchor comprises a slot formed in the bridge post anchor.

26. A method according to claim 16 wherein the second portion of the suture has a needle attached thereto.

27. A method according to claim 16 wherein the suture comprises a third portion secured to the body and a fourth portion residing free of the body and adapted to be passed soft tissue which is to be secured to the bone, and further wherein the system further comprises a second bridge post anchor for securing the fourth portion of the suture to the bone.

28. A method according to claim 16 wherein the capture element of the bridge post anchor comprises a suture spool movably mounted to the body of the bridge post anchor.

29. A method according to claim 28 wherein the suture spool is rotatable so as to cinch the suture.

30. A method according to claim 28 wherein the suture spool is longitudinally movable relative to the body so as to cinch the suture.

31. A system for attaching soft tissue to bone, the system comprising:
a center post anchor comprising a body having a distal end and a proximal end, a pair of legs extending distally from the distal end of the body and separated by a slot, the legs tapering outwardly along their length so that the center post anchor has a diameter at the legs which is larger than the diameter at the body, with the legs being inwardly compressible, and a suture attached to the proximal end of the body, the suture having at least one free end associated therewith; and
at least one bridge post anchor comprising a body having a distal end and a proximal end, and a pair of legs extending from the distal end of the body and separated by a slot, the legs tapering outwardly along their length so that the bridge post anchor has a diameter at the legs which is larger than the diameter at the body, with the legs being inwardly compressible;
such that the center post anchor can be secured in a hole in a bone by compressing its legs inwardly, deploying the center post anchor in the bone and releasing its legs so that they thereafter engage the bone, whereby to secure the suture to the bone;
and further such that the bridge post anchor can capture the free end of the suture to bone by positioning the free end of the suture in the slot, compressing the legs of the bridge post anchor inwardly, positioning the bridge post anchor in a hole in the bone, and releasing its legs so that they thereafter engage the bone.

32. A system according to claim 12 wherein the first portion of the suture, the second portion of the suture, the third portion of the suture and the fourth portion of the suture are all formed integral with one another so as to constitute a single filament.

33. A system according to claim 12 wherein the first portion of the suture and the second portion of the suture are formed integral with one another so as to constitute a first filament, and further wherein the third portion of the suture and the fourth portion of the suture are formed integral with one another so as to constitute a second filament, with the first filament and the second filament being separate from one another.

34. A method according to claim 16 wherein the first portion of the suture, the second portion of the suture, the third portion of the suture and the fourth portion of the suture are all formed integral with one another so as to constitute a single filament.

35. A system according to claim 31 wherein the first portion of the suture and the second portion of the suture are formed integral with one another so as to constitute a first filament, and further wherein the third portion of the suture and the fourth portion of the suture are formed integral with one another so as to constitute a second filament, with the first filament and the second filament being separate from one another.
